SEQUENCE LISTING (14 sequences)

SEQ ID NO: 1 - DNA, 22 bp, Artificial
  Synthetic
uacccuguagaaccgaauuugu

SEQ ID NO: 2 - DNA, 24 bp, Artificial
  Synthetic
uguaaacauccccgacuggaagcu

SEQ ID NO: 3 - DNA, 21 bp, Artificial
  Synthetic
caaagugcuguucgugcaggu

SEQ ID NO: 4 - DNA, 20 bp, Artificial
  Synthetic
ugagaugaagcacuguagcu

SEQ ID NO: 5 - DNA, 22 bp, Artificial
  Synthetic
aacauucaacgcugucggugag

SEQ ID NO: 6 - DNA, 22 bp, Artificial
  Synthetic
uuuggcaaugguagaacucaca

SEQ ID NO: 7 - DNA, 21 bp, Artificial
  Synthetic
ugcggggcuagggcuaacagc

SEQ ID NO: 8 - DNA, 23 bp, Artificial
  Synthetic
uacccuguagaaccgaauuugug

SEQ ID NO: 9 - DNA, 22 bp, Artificial
  Synthetic
uguaaacauccccgacuggaag

SEQ ID NO: 10 - DNA, 23 bp, Artificial
  Synthetic
caaagugcuguucgugcagguag

SEQ ID NO: 11 - DNA, 21 bp, Artificial
  Synthetic
ugagaugaagcacuguagcuc

SEQ ID NO: 12 - DNA, 23 bp, Artificial
  Synthetic
aacauucaacgcugucggugagu

SEQ ID NO: 13 - DNA, 24 bp, Artificial
  Synthetic
uuuggcaaugguagaacucacacu

SEQ ID NO: 14 - DNA, 22 bp, Artificial
  Synthetic
ugcggggcuagggcuaacagca

(19) United States
(12) Patent Application Publication
(10) Pub. No.: US 2016/0369344 A1
(43) Pub. Date: Dec. 22, 2016

(54) Detecting MicroRNA and Iso-MicroRNA to Evaluate Vascular and Cardiac Health

(71) Applicant: Emory University, Atlanta, GA (US)

(72) Inventors: Charles Searles, Atlanta, GA (US); Amanda Marie James, Decatur, GA (US)

(21) Appl. No.: 15/184,029

(22) Filed: Jun. 16, 2016

Related U.S. Application Data
(60) Provisional application No. 62/180,397, filed on Jun. 16, 2015.

(51) Int. Cl. C12Q 1/68

(52) U.S. Cl. CPC C12Q 1/6883; C12Q 2600/178; C12Q 2600/118

(57) ABSTRACT

This disclosure relates to diagnosing or evaluating the state of vascular or cardiac health of a subject by detecting alterations in the microRNA and/or iso-microRNA profile contained in a sample derived from the subject. Typically, the sample is derived from blood such as a plasma sample or portions further isolated therefrom. In certain embodiments, the sample is plasma from which microvesicles have been removed or the sample is enriched with blood derived microvesicles.

### CROSS-REFERENCE TO RELATED APPLICATIONS

This application claims the benefit of U.S. Provisional Application No. 62/180,397 filed Jun. 16, 2015. The entirety of this application is hereby incorporated by reference for all purposes.

### STATEMENT REGARDING FEDERALLY FUNDED RESEARCH

This invention was made with government support under the Merit Award I01 B00070 awarded the U.S. Department of Veterans Affairs and grant HHSN268201000043C from the National Institute of Health (NIH) and National Heart, Lung, and Blood Institute (NHLBI). The government has certain rights in the invention.

### INCORPORATION-BY-REFERENCE OF MATERIAL SUBMITTED AS A TEXT FILE VIA THE OFFICE ELECTRONIC FILING SYSTEM (EFS-WEB)

The Sequence Listing associated with this application is provided in text format in lieu of a paper copy, and is hereby incorporated by reference into the specification. The name of the text file containing the Sequence Listing is 14144US_ST25.txt. The text file is 3 KB, was created on Jun. 16, 2016, and is being submitted electronically via EFS-Web.

### BACKGROUND

Cardiovascular disease continues to be a major public health problem worldwide as individuals continue to experience unexpected heart attacks and succumb to cardiovascular disease. Therefore, there is a need to identify improved therapies and methods to diagnose and monitor cardiac and vascular health.

MicroRNAs (miRNAs) are non-coding RNAs that modulate gene expression typically by targeting mRNAs. MicroRNAs are generated as a family of related isomers that typically differ by a few bases at the 5′ and 3′ end of the miRNA, termed iso-miRs. Cloonan et al. report that miRNAs and iso-miRs function cooperatively to target common biological pathways. Genome Biol., 2011, 15, R126. Manzano et al. report that miRNA 5′-end variation sometimes leads to differential targeting. RNA 2015. 21: 1606-1620. MicroRNA may be packed into extracellular vesicles such as microvesicles contained in whole blood. Extracellular miRNAs have also been identified in high-density lipoprotein (HDL) or bound by the AGO2 protein.

Weber et al. report microRNA expression profiles in patients with coronary artery disease (CAD). Cardiol Res Pract. 2011; 2011: 532915. See also U.S. Patent Application Publication Number 2013/0289141.

Kanhai et al. report microvesicle protein levels are associated with increased risk for future vascular events and mortality in patients with clinically manifest vascular disease. Int J Cardiol. 2013, 168(3):2358-63.

Finn et al. report coronary heart disease alters intercellular communication by modifying microparticle-mediated microRNA transport. FEBS Lett. 2013, 587(21): 3456-3463.

Feinberg & Moore report on the regulation of atherosclerosis by microRNA. Circ Res. 2016, 118(4):703-20.

Jansen et al. report microRNA expression in circulating microvesicles predicts cardiovascular events in patients with coronary artery disease. J Am Heart Assoc. 2014; 3: e001249

References cited herein are not an admission of prior art.

### SUMMARY

This disclosure relates to diagnosing or evaluating the state of vascular or cardiac health of a subject by detecting alterations in the microRNA and/or iso-microR profile contained in a sample derived from the subject. Typically, the sample is derived from blood such as a plasma sample or portions further isolated therefrom. In certain embodiments, the sample is plasma from which microvesicles have been removed or the sample is enriched with plasma derived microvesicles.

In certain embodiments, the methods disclosed herein will aid in the diagnosis or treatment of, or indicate an increased risk of developing, or monitoring the status of a cardiovascular disease or related conditions such as coronary artery disease, peripheral artery disease, atherosclerosis, ischemia, angina, myocardial infarct, arrhythmia, ischemic stroke, hemorrhagic stroke, leg pain, cramps, hypertension, heart failure, aneurysm, renal artery disease, Reynaud's phenomenon, Buerger's disease, peripheral venous disease, varicose veins, blood clots, deep vein thrombosis, or lymphedema.

In certain embodiments, the disclosure relates to methods for diagnosing a subject as being at risk or having vascular, cardiovascular disease, or coronary artery disease, or of having higher concentrations of whole blood circulating microvesicles or other extracellular vesicles, or of having increased likelihood of having or having had a myocardial infarction comprising measuring in a sample the presence of one or more of the following microRNA (miRNA) and/or iso-microRNA (iso-miR) selected from miR-10b, miR-30d, miR-93, miR-143, miR-181a, miR-182, miR-744, or combinations thereof and correlating an increase or decrease in the amount of miRNA and/or iso-miR compared to a normal or reference value as being at risk or having cardiovascular disease or significant coronary artery disease, or of high plasma microvesicles, or increased likelihood of having or having had a myocardial infarction.

In certain embodiments, the miRNA and/or iso-miR is measured in a sample selected from whole plasma, microvesicles isolated from plasma, or plasma from which microvesicles have been removed.

In certain embodiments, the miRNA and/or iso-miR are iso-mR-93 and iso-miR-181a, wherein an increase of iso-miR-93 in whole plasma and a decrease of iso-miR181a in microvesicles isolated from plasma indicates having cardiovascular disease.

In certain embodiments, the miRNA and/or iso-miR are iso-miR-10b and iso-miR-93, wherein an increase of iso-miR-10b in whole plasma and a decrease of iso-miR-93 in microvesicles isolated from plasma indicates having high plasma microvesicles.

In certain embodiments, the miRNA and/or iso-miR are iso-miR-10b and iso-miR-93, wherein no change of iso-miR-10b in plasma from which microvesicles have been removed and a decrease of iso-miR-93 in microvesicles isolated from plasma indicates an increased likelihood of having had a myocardial infarction.

In certain embodiments, the miRNA and/or iso-miR measurements or correlations are recorded on a computer or computer readable medium. In certain embodiments, the method further comprises recording the similarities or differences to a normal or reference value on a computer readable medium. In certain embodiments, the miRNA and/or iso-miR measurements or correlations are displayed on a digital display. In certain embodiments, the methods disclosed herein further comprises the step of communicating the measurements or correlations to a medical professional or the subject.

In certain embodiments, the disclosure relates to a therapeutic strategy of identifying a subject is having or is at risk of a cardiovascular conditions using methods disclosed herein and administering a therapeutic agent to the subject based on the diagnosis.

### BRIEF DESCRIPTION OF THE FIGURES

FIG. 1 schematically illustrates embodiments of methods disclosed herein.

FIG. 2A shows data indicating a correlation of cardiovascular disease severity (CAD versus RF) with microvesicle counts. Cardiovascular disease has been noted to alter the abundance of circulating microvesicles (MV). Statistical modeling was performed based on cardiovascular disease severity bases on angiogram results: [Gensini Score] (RF vs CAD) and microvesicle counts.

FIG. 2B shows data indicating microvesicle counts correlate with myocardial infarction. Microvesicle counts in relation to the occurrence of a cardiac event (No MI vs MI). Together, these data indicate a relationship between angiogram results, cardiac events and MV counts as an indicator of disease progression.

FIG. 3 shows data indicating predominance of iso-miRs relative to consensus or archetypal miRNAs for specific miRNAs in RNA sequencing experiment. Deep sequencing analysis was performed on pooled blood samples matched by angiogram results, age, gender, and race. The percentage of iso-miR relative to consensus miRNA was calculated by dividing iso-miR reads by the total number of reads for a particular miRNA (consensus miRNA plus iso-miR).

FIG. 4 shows qRT-PCR data on the concentration (femtomolar) of different iso-miRs in plasma from Cohort 1 (risk factor-blue versus CAD-red). Each isomiR has a uniquely designed and validated RT/PCR Primer set.

FIG. 5A shows data indicating changes in iso-miR levels is altered by coronary artery disease severity. The arrows indicate relative concentration differences of certain biomarkers between isomiR in whole plasma, plasma w/o MVs and MVs when the patients were stratified by angiogram results. The arrows indicate increased (up arrow) or decreased (down arrow) concentration of iso-miRs in patients indicated by header relative to the other group of patients. For example, in whole plasma, isomiR-10b has increased concentration in CVD patients relative to RF patients and vice versa.

FIG. 5B shows data when the patients were stratified by MV Count. Arrows show relative differences in isomiR concentrations for patients with high MV counts versus low MV counts.

FIG. 5C shows data when the patients were stratified by cardiac events. Arrows show relative differences in iso-miR concentrations for patients with MI versus those who did not have MI.

FIG. 6 shows data where random forest modeling was used to assess the accuracy of combinations of two iso-miRs (isomiR-93 and 181a or isomiR-10b and -93) in predicting significant coronary artery disease or MI. Iso-miRs were measured in different plasma fractions (whole plasma, plasma w/o MVs or MVs).

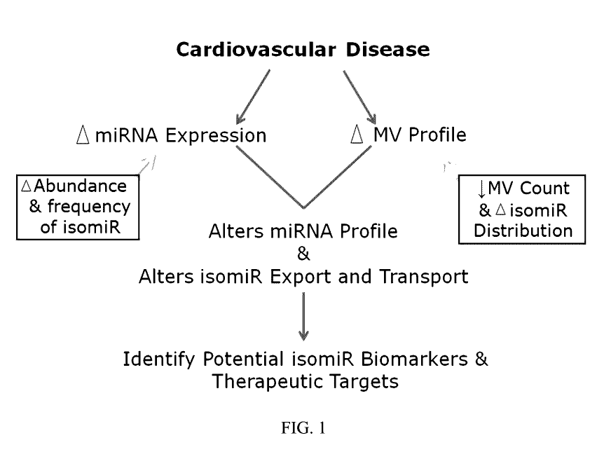

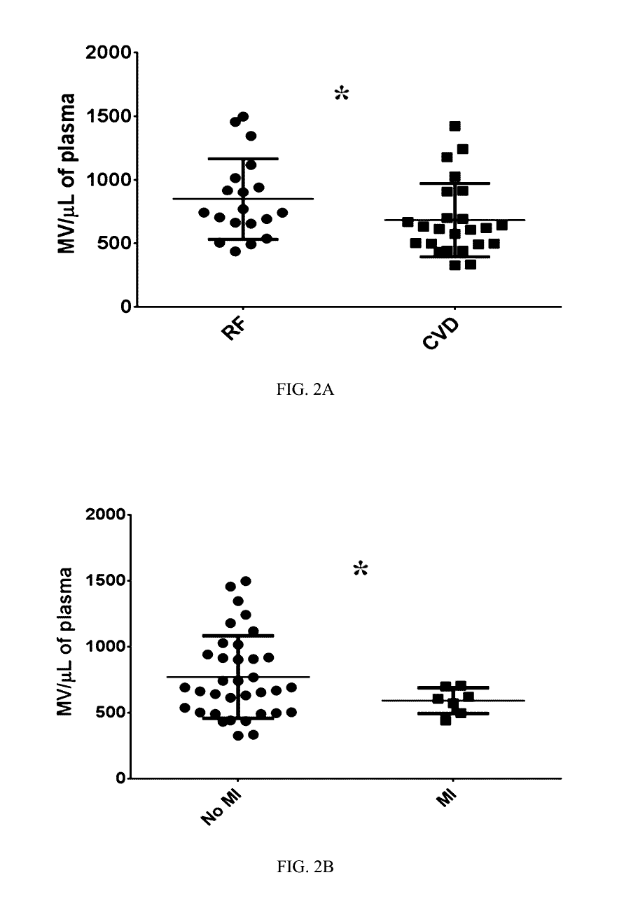

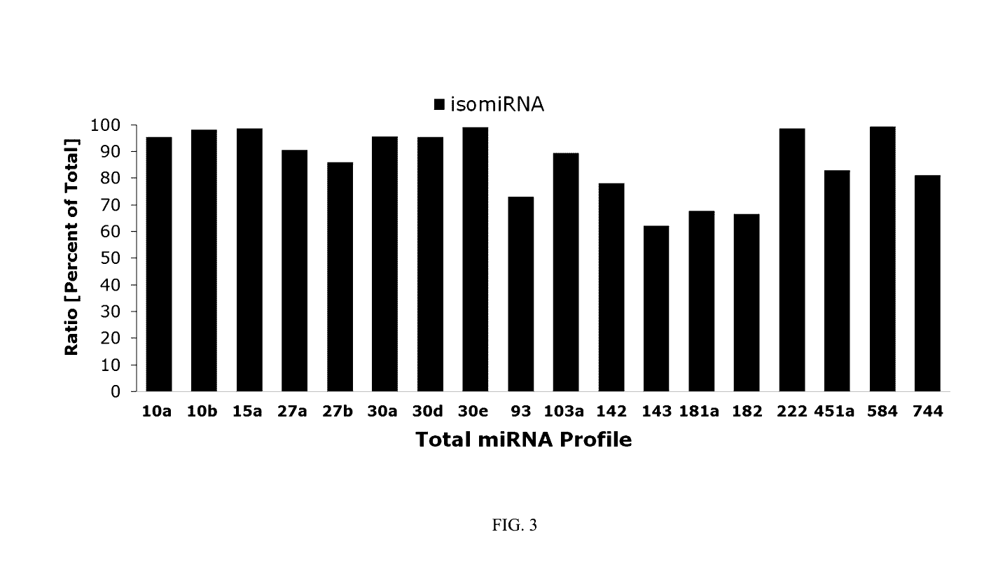

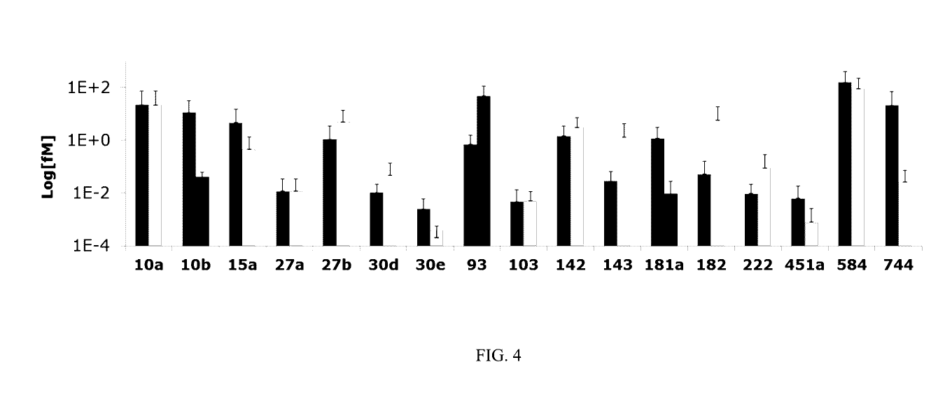

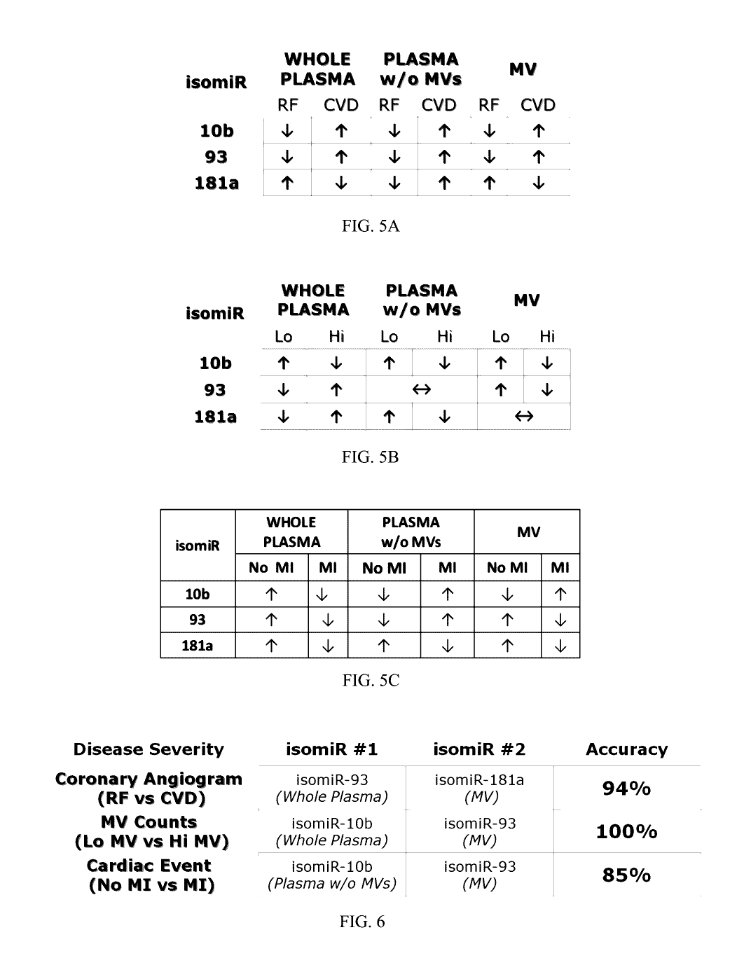

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

In the claims appended hereto, the term “a” or “an” is intended to mean “one or more,” and the term “comprise” and variations thereof such as “comprises” and “comprising,” when preceding the recitation of a step or an element, are intended to mean that the addition of further steps or elements is optional and not excluded.

The term “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) mammals, non-human mammals, primates, non-human primates, rodents, birds, reptiles, amphibians, fish, and any other animal. The subject can be a mammal such as a primate or a human patient.

The term “sample” refers to any mixture of biological materials derived from a subject, e.g., bodily fluids, whole blood, serum, plasma, tissue, skin, saliva, urine, stool, tears, amniotic fluid, breast milk etc. Biological samples may be obtained from animals (including humans) and encompass fluids, solids, tissues, and gases.

“Detecting” or like terms refers to measuring or otherwise determining an object or magnitude to be detected. For example, detection can refer to determining the presence or absence of an object or to determining or measuring a magnitude, such as a level, amount, or size of an object.

The term “hybridize” and the like, refers to a sequence driven interaction between at least two nucleic acid molecules, such as a primer or a probe and an RNA. Sequence driven interaction means an interaction that occurs between two nucleotides or nucleotide analogs or nucleotide derivatives in a nucleotide specific manner. For example, G interacting with C or A interacting with T are sequence driven interactions. The hybridization of two nucleic acids is affected by a number of conditions and parameters known to those of skill in the art. For example, the salt concentrations, pH, and temperature of the reaction all affect whether two nucleic acid molecules will hybridize.

“Level” and “amount” of an object can refer to, for example, a number or mass of the object, such as a dozen eggs or 100 mg of a molecule or to a concentration, such a molarity or grams per liter of the object or a molecule. Reference herein to level(s) or amount(s) in control subjects, reference subjects, normal subjects, or a combination should be understood to include the use of level(s) or amount(s) in one control subject, multiple control subjects, one reference subject, multiple reference subjects, one normal subject, multiple normal subjects, or any combination of these.

As used herein, a “normal subject” refers to a subject that does not have the relevant condition or disease.

As used herein, a “reference subject” refers to a subject that serves as a reference for the measurement to be made. For example, a reference subject can be a normal subject (when comparing for alterations or abnormal levels or amounts) or an affected subject (when comparison to one form or type of the disease or condition is indicated).

As used herein, a “reference level” refers to a level or amount that serves as a reference for the measurement to be made. For example, a reference level can be a normal level or amount (from a normal subject, normal tissue and/or normal body fluid), a control level or amount (from, for example, a normal subject, normal tissue and/or normal body fluid, or an affected subject, affected tissue and/or affected body fluid).

“Collecting” or like terms refers to obtaining, extracting, removing, or otherwise separating something from a starting location. For example, collecting tissue, a body fluid, a sample, a tissue sample, a body fluid sample, etc. from a subject refers to obtaining, extracting, removing, or otherwise separating the tissue, body fluid, sample, tissue sample, body fluid sample, etc. from the subject.

“Altering” or like terms refers to transforming or combining an object. For example, altering tissue, a body fluid, a sample, a tissue sample, a body fluid sample, microRNAs, etc. can refer to, for example, combining the tissue, body fluid, sample, tissue sample, body fluid sample, microRNA, etc. with compositions that transform the tissue, body fluid, sample, tissue sample, body fluid sample, microRNAs, etc. or produce a signal from the tissue, body fluid, sample, tissue sample, body fluid sample, microRNAs, etc.

The term “serum” refers to a product of whole blood manipulated under conditions such that the majority of clotting factors and cells have been substantially removed. In a typical procedure, whole blood is allowed to clot by leaving it at room temperature, which typically takes about 15-30 minutes. One can remove the clot by centrifuging at 1,000-2,000×g for 10 minutes in a refrigerated centrifuge. Serum is the resulting supernatant.

The term “plasma” refers to a product of whole blood manipulated under conditions such that the majority of cells have been substantially removed but maintains clotting factors. Plasma may or may not contain platelets. In a typical procedure, whole blood is treated with an anticoagulant and the cells are removed from plasma by centrifugation for 10 minutes at 1,000-2,000×g using a refrigerated centrifuge. Centrifugation for 15 minutes at 2,000×g depletes platelets in the plasma sample. Plasma is the resulting supernatant.

“Extracellular vesicles” refers to lipid membrane confined particles, e.g., having phospholipid bi-layer, which are shed from certain cells, such as blood cells. Extracellular vesicles may be characterized as microvesicles and exosomes. Microvesicles, otherwise known as microparticles, refers to extracellular vesicles with a size of about 100 nm-2 μm in diameter, and exosomes are smaller vesicles (less than 100 nm).

Extracellular vesicles or microvesicles may be removed from a bodily fluid by using higher and/or longer centrifugation conditions. Plasma from which microvesicles have been removed can typically be obtained by centrifuging at more than 10,000, or 15,000, or 16,500×g or more for 20 minutes or more. In a typical procedure, centrifugation of a bodily fluid at 100 to 2,000×g for 5 min or 10 min may be used to remove cell debris and for a further 90 min at 25,000 g to obtain the MVs. Centrifugation at greater than 100,000 g for more than 1 hour, will typically result is in mixture where both exosomes and microvesicles have been isolated from the plasma.

Panel of Seven Encapsulated and Nonencapsulated IsomiRNAs that Predict Coronary Atherosclerosis Severity and Cardiac Events

MicroRNAs (miRNAs) are small, non-coding RNAs that are post-transcriptional regulators of gene expression. They are recognized for their roles both as modulators of disease progression and as biomarkers of disease activity. While most miRNAs are intracellular, miRNAs have also been detected in various body fluids, including blood and urine. Extracellular miRNAs are highly stable, in large part due to their encapsulation in extracellular microvesicles or association with extracellular proteins. Because miRNAs are highly conserved across species and exhibit a dynamic range in response to disease states, extracellular miRNAs are felt to be promising biomarkers, a notion supported by recent clinical studies of colon cancer, breast cancer, hepatic cancer, kidney disease, neurologic disease, acute MI, and heart failure. Described herein are seven isomiRs (isomiR-10b, -30d, -93, -143, -181a, -182, -744) that one can detect in blood samples of patients with coronary artery disease. IsomiRs are variants of the archetypal or reference miRNAs annotated in miRBase, which is the main public repository for miRNA sequence data. IsomiRs differ from their archetypal miRNA by one to three nucleotides, commonly at the 3′ or 5′ end of the miRNA sequence, and their existence has only been recognized because of recent advances in RNA sequencing technology.

The iso-miRs are: 1) more abundant in human blood than their archetypal miRNAs; 2) predictive of coronary artery disease (CAD) severity; and 3) predictive of myocardial infarction (MI). Our strategy was to measure our isomiR panel in different plasma fractions: 1) whole plasma; 2) the microvesicle (MV) fraction of plasma (the pellet after plasma centrifugation at 16,500×g for 20 minutes); and 3) the non-microvesicle (non-MV) fraction of plasma (the supernatant after plasma centrifugation at 16,500×g for 20 minutes).

Data for 27 patients (10 cases—patients with significant CAD and 17 controls—patients with only risk factors for significant CAD) were obtained. Differences in iso-miR levels were found between cases and controls depending on whether iso-miRs were measured in whole plasma, microvesicle (MV) fraction, or non-MV fraction. Using random forest statistical modeling a combination of the abundances of the seven iso-miR in whole plasma, MV fraction and non-MV fraction was 84% accurate in predicting CAD versus risk factor (RF) status, whereas a combination of iso-miR levels in the different plasma fractions was 76% accurate in predicting which patients were going to have MI. A combination of two iso-miRs (isomiR-181a in MV fraction and -10b in non-MV fraction) was 90% accurate in predicting RF versus CAD, whereas a combination of iso-miR-93 in the MV fraction and -10b in the non-MV fraction was 85% accurate in predicting which patients were going to have MI.

Blood samples from an additional 44 patients (20 cases and 24 controls) were studied. Random forest statistical modeling of the abundance of the seven iso-miR panel in combinations of the three plasma fractions demonstrated that the iso-miR panel is 96% accurate in predicting CAD versus RF status and 82% accurate in predicting who will have MI.

In an analysis of a smaller panel of iso-miRs, a combination of two iso-miRs (isomiR-93 in whole plasma and isomiR-181a in the MV fraction) was 94% accurate in predicting CAD versus RF, whereas a combination of isomiR-10b in the non-MV fraction and isomiR-181a in the MV fraction was 90% accurate in predicting CAD versus RF status. In terms of predicting who was going to have MI, a combination of isomiR-10b in the non-MV fraction and isomiR-93 in the MV fraction was 85% accurate, and a combination of isomiR-93 in whole plasma and isomiR-181a in the non-MV fraction was 83% accurate.

The iso-miR panel is useful in helping clinicians risk stratify their patients otherwise have risk factors for CAD. For example, iso-miR abundance in different plasma fractions could influence whether a clinician will send a patient for non-invasive testing or straight to invasive assessment of CAD in the cardiac cath lab. Iso-miR abundance could also influence how aggressive medical therapy should be for a particular patient, especially when there may be relative contraindications to certain medications.

### Methods

This disclosure relates to evaluating the state of vascular or cardiac health of a subject by evaluating the microRNA (miRNA) and/or iso-microRNA (iso-miR) profile contained in a sample derived from the subject. In certain embodiments, the sample is derived from blood such as a plasma or serum sample or portions further isolated therefrom. In certain embodiments, the sample is enriched with blood derived microvesicles.

In certain embodiments, the methods disclosed herein will aid in the diagnosis or treatment of, or indicate an increased risk of developing, or monitoring a cardiovascular disease or related conditions such as peripheral artery disease, atherosclerosis, ischemia, transient ischemic attacks, angina, myocardial infarct, arrhythmia, ischemic stroke, hemorrhagic stroke, leg pain, cramps, hypertension, heart failure, aneurysm, renal artery disease, Reynaud's phenomenon, Buerger's disease, peripheral venous disease, varicose veins, blood clots, deep vein thrombosis, or lymphedema.

In certain embodiments, the disclosure relates to methods comprising detecting one or more target miRNA and/or iso-miR in tissue or a body fluid of a subject, where the level or amount of the one or more target miRNA and/or iso-miR indicates, for example, the risk, development, presence, severity, or a combination, of cardiovascular disease, circulating microvesicles, myocardial infarction, thoracic aortic aneurysm in the subject, of diastolic heart failure in the subject, of left ventricular hypertrophy in the absence of diastolic heart failure in the subject, of left ventricular remodeling in the subject, that the subject experienced ischemia-reperfusion, or combinations thereof.

In certain embodiments the disclosure contemplates methods for assessing cardiovascular disease, including cardiac failure, cardiac hypertrophy, thoracic aortic aneurysm, left ventricular remodeling using miRNA and/or iso-miR levels. The level of miRNA and/or iso-miR measured in a body fluid, such as plasma and serum, or in tissue, such as cardiac and aortic tissue

In certain embodiments, the disclosure relates to methods for diagnosing a subject as being at risk or having a cardiovascular disease, or of having high plasma microvesicles, or increased likelihood of having had a myocardial infarction comprising measuring in a sample the presence of one or more of the following iso-microRNA (iso-miR) selected from:

iso-miR-10b,
(SEQ ID NO: 1)
UACCCUGUAGAACCGAAUUUGU,
wherein the 3′ U is not followed by a G;
iso-miR-30d
(SEQ ID NO: 2)
UGUAAACAUCCCCGACUGGAAGCU;
iso-miR-93
(SEQ ID NO: 3)
CAAAGUGCUGUUCGUGCAGGU
wherein, the 3′ U is not followed by a AG;
iso-miR-143
(SEQ ID NO: 4)
UGAGAUGAAGCACUGUAGCU,
wherein, the 3′ U is not followed by a C;
iso-miR-181a
(SEQ ID NO: 5)
AACAUUCAACGCUGUCGGUGAG
wherein, the 3′ G is not followed by a U;
iso-miR-182
(SEQ ID NO: 6)
UUUGGCAAUGGUAGAACUCACA,
wherein, the 3′ A is not followed by a CU;
iso-miR-744
(SEQ ID NO: 7)
UGCGGGGCUAGGGCUAACAGC,
wherein, the 3′ C is not followed by a A;

and correlating an increase or decrease in the amount of miRNA or iso-miR compared to a normal or reference value as being at risk or having cardiovascular disease, or of high plasma microvesicles, or increased likelihood of having or having had a myocardial infarction or other ischemic event.

In certain embodiments, the microRNA (miRNA) and/or iso-microRNA (iso-miR) selected from:

iso-miR-10b,
(SEQ ID NO: 1)
UACCCUGUAGAACCGAAUUUGU,
wherein the 3′ U is not followed by a G,
has-miR-10b-5p,
(SEQ ID NO: 8)
UACCCUGUAGAACCGAAUUUGUG;,
iso-miR-30d
(SEQ ID NO: 2)
UGUAAACAUCCCCGACUGGAAGCU,
hsa-miR 30d-5p
(SEQ ID NO: 9)
UGUAAACAUCCCCGACUGGAAG;
iso-miR-93
(SEQ ID NO: 3)
CAAAGUGCUGUUCGUGCAGGU
wherein, the 3′ U is not followed by a AG,
hsa-miR-5p
(SEQ ID NO: 10)
CAAAGUGCUGUUCGUGCAGGUAG,
iso-miR-143
(SEQ ID NO: 4)
UGAGAUGAAGCACUGUAGCU,
wherein, the 3′ U is not followed by a C,
hsa-miR-143-3p
(SEQ ID NO: 11)
UGAGAUGAAGCACUGUAGCUC
iso-miR-181a
(SEQ ID NO: 5)
AACAUUCAACGCUGUCGGUGAG
wherein, the 3′ G is not followed by a U,
hsa-miR-181a-5p
(SEQ ID NO: 12)
AACAUUCAACGCUGUCGGUGAGU
iso-miR-182
(SEQ ID NO: 6)
UUUGGCAAUGGUAGAACUCACA,
wherein, the 3′ A is not followed by a CU,
hsa-miR-182-5p
(SEQ ID NO: 13)
UUUGGCAAUGGUAGAACUCACACU
iso-miR-744
(SEQ ID NO: 7)
UGCGGGGCUAGGGCUAACAGC,
wherein, the 3′ C is not followed by a A
hsa-miR-744-5p
(SEQ ID NO: 14)
UGCGGGGCUAGGGCUAACAGCA.

In certain embodiments, the miRNA and/or iso-miR is measured in a sample selected from plasma, microvesicles isolated from plasma, or plasma from which microvesicles have been removed.

In certain embodiments, the miRNA and/or iso-miR are iso-mR-93 and iso-miR-181a, wherein an increase of iso-miR-93 in whole plasma and a decrease of iso-miR181a in microvesicles isolated from plasma indicates having significant coronary artery disease or cardiovascular disease.

In certain embodiments, the miRNA and/or iso-miR are iso-miR-10b and iso-miR-93, wherein an increase of iso-miR-10b in whole plasma and a decrease of iso-miR-93 in microvesicles isolated from plasma indicates having high plasma microvesicles.

In certain embodiments, the miRNA and/or iso-miR are iso-miR-10 and iso-miR-93, wherein an increase of iso-miR-10b in plasma from which microvesicles have been removed and a decrease of iso-miR-93 in microvesicles isolated from plasma indicates an increased likelihood of having or having had a myocardial infarction.

In certain embodiments, the methods for assessing vascular health or disease state, include evaluating for cardiac failure, cardiac hypertrophy, thoracic aortic aneurysm, or ventricular remodeling using miRNA and/or iso-miR levels. The level of miRNA and/or iso-miR can be measured in a body fluid, such as plasma and serum, or in tissue, such as cardiac and aortic tissue.

In some forms, a set of miRNA and/or iso-miR is detected and if a threshold percentage or more of the set of miRNA and/or iso-miR has an indicated level or amount, the subject can be assessed for a disease or condition. For example, if a threshold percentage or more of a set of miRNA and/or iso-miR has a level or amount of less than or greater than (depending on the individual miRNA and/or iso-miR) than the level or amount in control subjects, reference subjects, normal subjects, or a combination, it indicates a risk, presence, severity, or a combination of a cardiovascular disease. In some forms, the threshold percentage can be on or about 60%, 65%, 70%, 75%, 80%, 85%, 87%, 88%, 89%, 90%, 91%, 92%, 93%, 94%, 95%, 96%, 97%, 98%, 99%, or more of the target miRNA and/or iso-miR.

The decrease in the level or amount of the one or more target miRNA and/or iso-miR can be determined by detecting the level or amount of the one or more target miRNA and/or iso-miR at different times. The level or amount of the one or more target miRNA and/or iso-miR at one or more of the different times can be greater than the level or amount in control subjects, reference subjects, normal subjects, or a combination.

The body fluid can be, for example, blood, plasma, serum, or lymphatic fluid. The plurality of different times at which the one or more miRNA and/or iso-miR are detected can comprise two or more times separated by 1, 2, 3, 4, 5, 10, 15, 20, 23, 24, 25, 26, 27, 28, 30, 35, 40, 45, 50, 55, 60, 62, 65, 70, 75, 80, 85, 86, 87, 88, 89, and 90 days. The level of the one or more target miRNA and/or iso-miR can comprise the measured level of the one or more target miRNA and/or iso-miR normalized to the measured level of a reference miRNA and/or iso-miR in the body fluid.

The level of the one or more target miRNA and/or iso-miR can comprise the measured level of the one or more target miRNA and/or iso-miR expressed as the fold difference of the measured level of the one or more target miRNA and/or iso-miR to the measured level of the one or more target miRNA and/or iso-miR in a reference subject. The level of the one or more target miRNA and/or iso-miR can comprise the measured level of the one or more target miRNA and/or iso-miR normalized to the measured level of a reference miRNA and/or iso-miR in the body fluid expressed as the fold difference of the normalized level of the one or more target miRNA and/or iso-miR to the measured level of the one or more target miRNA and/or iso-miR in the same body fluid of reference subject normalized to the measured level of a reference miRNA and/or iso-miR in the body fluid of the reference subject.

In certain embodiments, the level of the one or more target miRNA and/or iso-miR in a reference subject can be measured at the same time as the level of the one or more target miRNA and/or iso-miR is measured in the subject. The level of the one or more target miRNA and/or iso-miR in a reference subject can be measured at a different time than the level of the one or more target miRNA and/or iso-miR is measured in the subject. The level of the one or more target miRNA and/or iso-miR in a reference subject can be a reference level.

In certain embodiments, the plurality of different times can comprise two or more times 1, 2, 3, 4, 5, 10, 15, 20, 25, 28, 30, 35, 40, 45, 50, 55, 60, 65, 70, 75, 80, 85, and 90 days following a known or suspected myocardial infarction in the subject. The temporal pattern of the level of the one or more target miRNA and/or iso-miR can indicate that the subject suffered a myocardial infarction. The temporal pattern of the level of the one or more target miRNA and/or iso-miR can indicate how long ago the subject suffered the myocardial infarction.

In certain embodiments, the disclosure relates to a therapeutic strategy of identifying a subject is having, or is at risk of, or monitoring a cardiovascular conditions using methods disclosed herein and administering a therapeutic agent or agents such as an anticoagulant, antiplatelet agent, angiotensin-converting enzyme (ACE) inhibitor, angiotensin II receptor blocker, angiotensin-receptor neprilysin inhibitor, beta-adrenergic blocker, calcium channel blocker, cholesterol-lowering medication, digoxin, digitoxin, diuretic, vasodilator, or combinations thereof to the subject based on the diagnosis or monitoring activities.

In certain embodiments, the agent is an anticoagulant such as rivaroxaban, dabigatran, apixaban, heparin or variants, warfarin, or combinations thereof.

In certain embodiments, the agent is an antiplatelet agent such as aspirin, clopidogrel, dipyridamole, prasugrel, ticagrelor, or combinations thereof.

In certain embodiments, the agent is an angiotensin-converting enzyme (ACE) inhibitor such as benazepril, captopril, enalapril, fosinopril, lisinopril, moexipril, perindopril, quinapril, ramipril, trandolapril, or combinations thereof.

In certain embodiments, the agent is an angiotensin II receptor blocker such as candesartan, eprosartan, irbesartan, losartan, telmisartan, valsartan, or combinations thereof.

In certain embodiments, the agent is an angiotensin-receptor neprilysin inhibitor sush as sacubitril, valsartan, or combinations thereof.

In certain embodiments, the agent is a beta-adrenergic blocking agents such as acebutolol, atenolol, betaxolol, bisoprolol, hydrochlorothiazide, metoprolol, nadolol, propranolol, sotalol, or combinations thereof.

In certain embodiments, the agent is a calcium channel blocker such as amlodipine, diltiazem, felodipine, nifedipine, nimodipine, nisoldipine, verapamil, or combinations thereof.

In certain embodiments, the agent is a cholesterol-lowering medication such as atorvastatin, rosuvastatin, lovastatin, ezetimibe, simvastatin, or combinations thereof.

In certain embodiments, the agent is a diuretic such as amiloride, bumetanide, chlorothiazide, chlorthalidone, furosemide, hydro-chlorothiazide, indapamide, spironolactone, or combinations thereof.

In certain embodiments, the agent is a vasodilator such as isosorbide dinitrate, nesiritide, hydralazine, nitrates, minoxidil, or combinations thereof.

### Detection and Quantitation

To aid in detection and quantitation of miRNA and/or iso-miR, detection labels can be incorporated into detection probes or detection molecules or directly incorporated into amplified nucleic acids. As used herein, a detection label is any molecule that can hybridize or otherwise be associated with nucleic acid, directly or indirectly, and which results in a measurable, detectable signal, either directly or indirectly. Many such labels are known to those of skill in the art. Examples of detection labels suitable for use in the disclosed method are radioactive isotopes, fluorescent molecules, phosphorescent molecules, enzymes, antibodies, and ligands.

Additional labels of interest include those that provide for signal only when the probe with which they are associated is specifically bound to or hybridize with a target molecule, where such labels include: “molecular beacons” as described in Tyagi & Kramer, Nature Biotechnology (1996) 14:303. Other labels of interest include those described in U.S. Pat. No. 5,563,037; WO 97/17471 and WO 97/17076.

Labeled nucleotides are a useful form of detection label for direct incorporation into expressed nucleic acids during synthesis. Examples of detection labels that can be incorporated into nucleic acids include nucleotide analogs such as BrdUrd (5-bromodeoxyuridine, Hoy and

Schimke, Mutation Research 290:217-230 (1993)), aminoallyldeoxyuridine (Henegariu et al., Nature Biotechnology 18:345-348 (2000)), 5-methylcytosine (Sano et al., Biochim. Biophys. Acta 951:157-165 (1988)), bromouridine (Wansick et al., J. Cell Biology 122:283-293 (1993)) and nucleotides modified with biotin (Langer et al., Proc. Natl. Acad. Sci. USA 78:6633 (1981)) or with suitable haptens such as digoxygenin (Kerkhof, Anal. Biochem. 205:359-364 (1992)). Suitable fluorescence-labeled nucleotides are Fluorescein-isothiocyanate-dUTP, Cyanine-3-dUTP and Cyanine-5-dUTP (Yu et al., Nucleic Acids Res., 22:3226-3232 (1994)). A preferred nucleotide analog detection label for DNA is BrdUrd (bromodeoxyuridine, BrdUrd, BrdU, BUdR, Sigma-Aldrich Co). Other useful nucleotide analogs for incorporation of detection label into DNA are AA-dUTP (aminoallyl-deoxyuridine triphosphate, Sigma-Aldrich Co.), and 5-methyl-dCTP (Roche Molecular Biochemicals). A useful nucleotide analog for incorporation of detection label into RNA is biotin-16-UTP (biotin-16-uridine-5′-triphosphate, Roche Molecular Biochemicals). Fluorescein, Cy3, and Cy5 can be linked to dUTP for direct labeling. Cy3.5 and Cy7 are available as avidin or anti-digoxygenin conjugates for secondary detection of biotin- or digoxygenin-labeled probes.

Detection labels that are incorporated into nucleic acid, such as biotin, can be subsequently detected using sensitive methods well-known in the art. For example, biotin can be detected using streptavidin-alkaline phosphatase conjugate, which is bound to the biotin and subsequently detected by chemiluminescence of suitable substrates (for example, chemiluminescent substrate CSPD: disodium, 3-(4-methoxyspiro-[1,2,-dioxetane-3-2′-(5′-chloro)tricyclo[3.3.-1.1.sup.3,7]decane]-4-yl) phenyl phosphate). Labels can also be enzymes, such as alkaline phosphatase, soybean peroxidase, horseradish peroxidase and polymerases, that can be detected, for example, with chemical signal amplification or by using a substrate to the enzyme which produces light (for example, a chemiluminescent 1,2-dioxetane substrate) or fluorescent signal.

Molecules that combine two or more of these detection labels are also considered detection labels. Any of the known detection labels can be used with the disclosed probes, tags, molecules and methods to label and detect miRNA and/or iso-miR or nucleic acid produced in the disclosed methods. Methods for detecting and measuring signals generated by detection labels are also known to those of skill in the art. For example, radioactive isotopes can be detected by scintillation counting or direct visualization; fluorescent molecules can be detected with fluorescent spectrophotometers; phosphorescent molecules can be detected with a spectrophotometer or directly visualized with a camera; enzymes can be detected by detection or visualization of the product of a reaction catalyzed by the enzyme; antibodies can be detected by detecting a secondary detection label coupled to the antibody. As used herein, detection molecules are molecules which interact with a compound or composition to be detected and to which one or more detection labels are coupled.

So long as their relevant function is maintained, primers, probes, and any other oligonucleotides and nucleic acids can be made up of or include modified nucleotides (nucleotide analogs). Many modified nucleotides are known and can be used in oligonucleotides and nucleic acids. A nucleotide analog is a nucleotide which contains some type of modification to either the base, sugar, or phosphate moieties. Modifications to the base moiety would include natural and synthetic modifications of A, C, G, and T/U as well as different purine or pyrimidine bases, such as uracil-5-yl, hypoxanthin-9-yl (I), and 2-aminoadenin-9-yl. A modified base includes but is not limited to 5-methylcytosine (5-Me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted racils and cytosines, 7-methylguanine and 7-methyladenine, 8-azaguanine and 8-azaadenine, 7-deazaguanine and 7-deazaadenine and 3-deazaguanine and 3-deazaadenine. Additional base modifications can be found for example in U.S. Pat. No. 3,687,808, Englisch et al., Angewandte Chemie, International Edition, 1991, 30, 613, and Sanghvi, Y. S., Chapter 15, Antisense Research and Applications, pages 289-302, Crooke, S. T. and Lebleu, B. ed., CRC Press, 1993. Certain nucleotide analogs, such as 5-substituted pyrimidines, 6-azapyrimidines and N2, N-6 and 0-6 substituted purines, including 2-aminopropyladenine, 5-propynyluracil and 5-propynylcytosine. 5-methylcytosine can increase the stability of duplex formation. Other modified bases are those that function as universal bases. Universal bases include 3-nitropyrrole and 5-nitroindole. Universal bases substitute for the normal bases but have no bias in base pairing. That is, universal bases can base pair with any other base. Base modifications often can be combined with for example a sugar modification, such as 2′-O-methoxyethyl, to achieve unique properties such as increased duplex stability.

Solid supports are solid-state substrates or supports with which molecules (such as probes) or other components used in, or produced by, the disclosed methods can be associated. Molecules can be associated with solid supports directly or indirectly. For example, probes can be bound to the surface of a solid support. An array is a solid support to which multiple probes or other molecules have been associated in an array, grid, or other organized pattern.

An array can include a plurality of molecules, compounds or probes immobilized at identified or predefined locations on the solid support. Each predefined location on the solid support generally has one type of component (that is, all the components at that location are the same). Alternatively, multiple types of components can be immobilized in the same predefined location on a solid support. Each location will have multiple copies of the given components. The spatial separation of different components on the solid support allows separate detection and identification.

Each of the components immobilized on the solid support can be located in a different predefined region of the solid support. The different locations can be different reaction chambers. Each of the different predefined regions can be physically separated from each other of the different regions. The distance between the different predefined regions of the solid support can be either fixed or variable. For example, in an array, each of the components can be arranged at fixed distances from each other, while components associated with beads will not be in a fixed spatial relationship. In particular, the use of multiple solid support units (for example, multiple beads) will result in variable distances.

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for detecting and measuring miRNA and/or iso-miR, the kit comprising amplification primers and detection probes. The kits also can contain enzymes and reaction solutions.

Disclosed are mixtures formed by performing or preparing to perform the disclosed method. For example, disclosed are mixtures comprising a body fluid and amplification primers, microRNA and amplification primers, and amplified microRNA and detection probes.

### Identifying, Recording, and Reporting

In certain embodiments, the disclosure relates to methods comprising identifying, recording, and reporting miRNA and/or iso-miR, diagnosis and therapeutic strategies related thereto. Disclosed are data structures used in, generated by, or generated from, the disclosed method. Data structures generally are any form of data, information, and/or objects collected, organized, stored, and/or embodied in a composition or medium. A temporal pattern of microRNA levels stored in electronic form, such as in RAM or on a storage disk, is a type of data structure. The disclosed method, or any part thereof or preparation therefor, can be controlled, managed, or otherwise assisted by computer control. Such computer control can be accomplished by a computer controlled process or method, can use and/or generate data structures, and can use a computer program. Such computer control, computer controlled processes, data structures, and computer programs are contemplated and should be understood to be disclosed herein.

In certain embodiments, the miRNA and/or iso-miR measurements or correlations are recorded on a computer or computer readable medium. In certain embodiments, the method further comprises recording the similarities or differences to a normal or reference value on a computer readable medium. In certain embodiments, the miRNA and/or iso-miR measurements or correlations are displayed on a digital display. In certain embodiments, the methods disclosed herein further comprises the step of communicating the measurements or correlations to a medical professional or the subject.

The disclosed methods include the determination, identification, indication, correlation, diagnosis, prognosis, etc. (which can be referred to collectively as “identifications”) of subjects, diseases, conditions, states, etc. based on measurements, detections, comparisons, analyses, assays, screenings, etc. For example, levels or amounts of miRNA and/or iso-miR can be used to identify the cardiac or vascular health of a subject, e.g., that subject have or are at risk of a cardiac disease, vascular disease, myocardial infarction. Such identifications are useful for many reasons. For example, and in particular, such identifications allow specific actions to be taken based on, and relevant to, the particular identification made. For example, diagnosis of a particular disease or condition in particular subjects (and the lack of diagnosis of that disease or condition in other subjects) has the very useful effect of identifying subjects that would benefit from treatment, actions, behaviors, etc. based on the diagnosis. For example, treatment for a particular disease or condition in subjects identified is significantly different from treatment of all subjects without making such an identification (or without regard to the identification). Subjects needing or that could benefit from the treatment will receive it and subjects that do not need or would not benefit from the treatment will not receive it.

Accordingly, also disclosed herein are methods comprising taking particular actions following and based on the disclosed identifications. For example, disclosed are methods comprising creating a record of an identification (in physical-such as paper, electronic, or other-form, for example). Thus, for example, creating a record of an identification based on the disclosed methods differs physically and tangibly from merely performing a measurement, detection, comparison, analysis, assay, screen, etc. Such a record is particularly substantial and significant in that it allows the identification to be fixed in a tangible form that can be, for example, communicated to others (such as those who could treat, monitor, follow-up, advise, etc. the subject based on the identification); retained for later use or review; used as data to assess sets of subjects, treatment efficacy, accuracy of identifications based on different measurements, detections, comparisons, analyses, assays, screenings, etc., and the like. For example, such uses of records of identifications can be made, for example, by the same individual or entity as, by a different individual or entity than, or a combination of the same individual or entity as and a different individual or entity than, the individual or entity that made the record of the identification. The disclosed methods of creating a record can be combined with any one or more other methods disclosed herein, and in particular, with any one or more steps of the disclosed methods of identification.

1. A method for diagnosing a subject as being at risk or having cardiovascular disease comprising measuring in a sample the presence of one or more of the following iso-microRNA (iso-miR) selected from:
iso-miR-10b,
(SEQ ID NO: 1)
UACCCUGUAGAACCGAAUUUGU,
wherein the 3′ U is not followed by a G;
iso-miR-30d
(SEQ ID NO: 2)
UGUAAACAUCCCCGACUGGAAGCU;
iso-miR-93
(SEQ ID NO: 3)
CAAAGUGCUGUUCGUGCAGGU
wherein, the 3′ U is not followed by a AG;
iso-miR-143
(SEQ ID NO: 4)
UGAGAUGAAGCACUGUAGCU,
wherein, the 3′ U is not followed by a C;
iso-miR-181a
(SEQ ID NO: 5)
AACAUUCAACGCUGUCGGUGAG
wherein, the 3′ G is not followed by a U;
iso-miR-182
(SEQ ID NO: 6)
UUUGGCAAUGGUAGAACUCACA,
wherein, the 3′ A is not followed by a CU;
iso-miR-744
(SEQ ID NO: 7)
UGCGGGGCUAGGGCUAACAGC,
wherein, the 3′ C is not followed by a A;
and correlating an increase or decrease in the amount of iso-miR compared to a normal or reference value as being at risk or having cardiovascular disease.

2. The method of claim 1, wherein the iso-miR is measured in a sample selected from whole plasma, microvesicles isolated from plasma, or plasma from which microvesicles have been removed.

3. The method of claim 2 wherein the iso-miR are iso-mR-93 and iso-miR-181a, wherein an increase of iso-miR-93 in whole plasma and an decrease of iso-miR181a in microvesicles isolated from plasma indicates having cardiovascular disease.

4. The method of claim 2 wherein the iso-miR are iso-miR-10b and iso-miR-93, wherein an increase of iso-miR-10b in whole plasma and an decrease of iso-miR-93 in microvesicles isolated from plasma indicates having high plasma microvesicles.

5. The method of claims 2, wherein the iso-miR are iso-miR-10b and iso-miR-93, wherein an increase of iso-miR-10b in plasma from which microvesicles have been removed and a decrease of iso-miR-93 in microvesicles isolated from plasma indicates an increased likelihood of having had a myocardial infarction.

6. The method of claim 2, wherein iso-miR measurements or correlations are recorded on a computer or computer readable medium.

7. The method of claim 6, wherein iso-miR measurements or correlations are displayed on a digital display.

8. The method of claim 7, wherein the method further comprises the step of communicating the measurements or correlations to a medical professional or the subject.

9. The method of claim 8, further comprising administering a therapeutic agent to the subject.
